(19) United States
(12) Patent Application Publication
(10) Pub. No.: US 2025/0352147 A1
(43) Pub. Date: Nov. 20, 2025

(54) PHYSIOLOGICAL MONITORING DEVICE FOR DRIVERS

(71) Applicant: SPINA CARE CORPORATION, Houston, TX (US)

(72) Inventor: Ivailo Zlatkov DACHOV, Sofia (BG)

(73) Assignee: SPINA CARE CORPORATION, Houston, TX (US)

(21) Appl. No.: 18/668,705

(22) Filed: May 20, 2024

(51) Int. Cl. A61B 5/00; A61B 5/0205; A61B 5/145

(52) U.S. Cl. CPC A61B 5/72; A61B 5/0205; A61B 5/14542; A61B 2503/22

(57) ABSTRACT

The present invention relates to physiological monitoring device and system coupled to a steering wheel for monitoring a subject such as a driver or a haulier. In particular, the present invention is directed to a physiological monitoring device configured to be biased against the steering wheel to expose a physiological monitor/sensor for a nominal contact against the palm of a driver to measure and monitor physiological parameters. The medical device of the present invention can measure or determine continuously, certain physiologically relevant biomarkers of a subject such as a driver or a haulier. More in particular, the present invention is directed to a physiological monitoring medical device configured to continuously measure and monitor physiologically relevant biomarkers such as pulse rate, heart rate, SpO₂ or blood pressure of the subject such as a driver or a haulier.

### FIELD OF THE INVENTION

The present invention is in the field of physiological health monitoring, and more particularly, sensors and computational devices that contact a body part to provide quantitative health measures.

### BACKGROUND OF THE INVENTION

Noninvasive, continuous and reliable physiological monitoring of for example pulse, SpO₂ and blood pressure, is vital to the healthcare of individuals. Noninvasive, continuous and reliable physiological monitoring of for example pulse, heart rate, SpO₂ or blood pressure, is vital to the healthcare of drivers and especially hauliers. Many noninvasive physiological monitoring systems, however, require that the device sensing the physiological parameters maintain an effective skin contact while at the same time relying on established approaches such as mathematical models, numerical simulations, empiric methods or comparative analysis, which deliver inconsistent and often inaccurate readings which can be misleading to physicians and patients alike. Moreover, most of the current noninvasive physiological monitoring devices and systems can be cumbersome e.g. ambulatory measurements of blood pressure using inflatable cuffs, and incapable of providing easy to use, continuous, accurate and reliable physiological parameters assessment which can be safely relied on.

Moreover, there is currently no physiological monitoring device which can measure, or determine continuously, in real-time, biomarkers such as health relevant biomarkers which can be used for example to monitor a subject such as a driver or a haulier.

As can be seen, there is an unmet need for a more accurate device configured to expose a physiological monitor with a nominal contact against the skin of the subject such as a driver or hauliers, for continuous, accurate and reliable physiological monitoring of health relevant biomarkers for example such as for instance pulse rate, heart rate, SpO₂ or blood pressure. This unmet need is at least in part addressed by the present invention.

### SUMMARY OF THE INVENTION

In one aspect of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel; wherein the biased first housing and the biased second housing expose the first monitoring interface and the second monitoring interface against a palm of a subject such as a driver or a haulier.

In some embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing expose the first monitoring interface and the second monitoring interface against a palm of a driver, and wherein operatively associated with the first monitoring interface and the second monitoring interface and contained in the first housing and the second housing are two or more physiological monitor units.

In some further embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing expose the first monitoring interface and the second monitoring interface against a palm of a subject, wherein operatively associated with the first monitoring interface and the second monitoring interface and contained in the first housing and the second housing are two or more physiological monitor units; and wherein the first monitoring interface and the second monitoring interface are operatively linked by a subsidiary module or a processor module.

In some further embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing expose the first monitoring interface and the second monitoring interface against a palm of a subject, wherein operatively associated with the first monitoring interface and the second monitoring interface and contained in the first housing and the second housing are two or more physiological monitor units; where the first monitoring interface and the second monitoring interface are operatively liked by a processor module or subsidiary module; and wherein the processor module or subsidiary module is coupled to the vehicle.

These and other features, aspects and advantages of the present invention will become better understood with reference to the following drawings, description, and claims.

### BRIEF DESCRIPTION OF THE DRAWINGS

FIG. 1. An in situ depiction of the medical device comprising first monitoring interface with sensor (1), second monitoring interface with sensor (2) and subsidiary/processor module (5). Connection (3 and 4) can be used as a conduit between first monitoring interface (3) second monitoring interface (4) and the subsidiary/processor module (5). Connection (6) can be used as a conduit between the processor module and the vehicle's (e.g. car or lorry) control systems (e.g. computer). The connectivity between the first monitoring interface and the subsidiary module with the second monitoring interface may be via Bluetooth, WiFi or the like.

FIG. 2. Schematic representation of the medical device comprising first monitoring interface with sensor, second monitoring interface with sensor and subsidiary/processor module configured to be coupled to a steering wheel.

FIG. 3. Schematic representation of the medical device comprising first monitoring interface with sensor (1), second monitoring interface with sensor (2), subsidiary/processor module (5) and connecting conduits (3, 4 and 6). The connectivity between the first monitoring interface and the subsidiary module with the second monitoring interface and/or the vehicle's control systems can be via Bluetooth, WiFi or the like.

FIG. 4. Steering wheel mounted medical device comprising first monitoring interface with sensor and second monitoring interface with sensor. The subsidiary/processor module is not visible. The connectivity between the first monitoring interface and the subsidiary/processor module and the second monitoring interface and the subsidiary/processor module and/or the vehicle's control systems (e.g. on-board computer) can be via Bluetooth, WiFi or the like. It is also contemplated that the vehicle's control systems (e.g. on-board computer) can be adapted or configured to perform the operations or functions of the subsidiary/processor module.

FIG. 5. Schematic representation of medical device system (a) readings or data collection (b), correlation between physiological parameter measurements such as pulse rate, SpO₂, blood pressure, capillary blood pulse wave and capillary blood pulse wave data, mathematical manipulation and software analysis of the data e.g. by vehicle's onboard computer system (c), transmittal and storage of the data on a Server/iCloud/vehicle's computer (d), and downstream uses of the collated physiological data (e).

FIG. 6. The medical device can be used to discern a delay in Pulse Transit Time (PTT) between both capillary blood pulse wave and arterial blood pulse wave. PTT has a direct link with changes in blood pressure which has an impact on safety and reliability of the driver or the haulier and the public.

### DETAILED DESCRIPTION OF THE INVENTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and the claims, the singular form “a,” “an” and “the” include singular and plural references unless the context clearly dictates otherwise. For example, the term “biomarker” includes a single or plurality of biomarkers.

Throughout the specification, when a portion, interface or housing is “connected” or “coupled” to another portion, such as first monitoring interface to subsidiary module, this includes not only a case of being “directly connected or coupled” but also a case of being “electrically connected” with another element interposed therebetween, as well as for example “WiFi connected”, “Bluetooth connected”, “computer connected”, “vehicle connected”, “vehicle computer connected”, “vehicle control systems connected”, “processor module” or the like.

As used herein, the term “palm” means the underside of a human hand such as a driver or haulier, also referred to as the broad palm or metacarpus consisting of the area between the five phalanges (i.e. finger bones) and the carpus (i.e. wrist joint). In the context of the present invention, it is particularly advantageous that, unlike other areas of the body, the skin of the palm is glabrous (i.e. hairless) and thus offers increased sensitivity. Preferably, the monitoring interface of the device of the present invention such as the first monitoring interface and the second monitoring interface, are exposed to or in communication or contact with the skin of the palm of the hand.

As used herein the term “subject” is interchangeable with the term “human” and means generally to a driver or an operator of a vehicle or a haulier.

Throughout the specification, when a member is said to be located “on” or “by” another member, constituent or unit, this includes not only a case in which the member, constituent or unit, is in contact with another member, constituent or unit, but also a case in which there is another member, constituent or unit between the two members, constituents or units or more. A member, constituent or unit may include for instance a monitoring interface, a housing, a processor/subsidiary module, computer, device or control system.

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a medical device, comprising: a first monitoring interface contained in a first housing and a second monitoring interface contained in a second housing; the first monitoring interface and the second monitoring interface being operatively linked; the first housing and the second housing being configured to be biased against a steering wheel; wherein the biased first housing and the biased second housing expose the first monitoring interface and the second monitoring interface against a palm of a driver.

The biasing component is configured to urge the first housing and the second housing against a steering wheel of a vehicle. As used herein the term “vehicle” means any type of vehicle such as a car, SUV (sports utility vehicle), VAN, lorry, truck, tractor, forklift, combine harvester, crane, a simulator or the like.

The first monitoring interface and the second monitoring interface are exposed against the skin of a driver and urge a nominal contact force between the first monitoring interface and the second monitoring interface and the palms of a driver.

In some embodiments, the first monitoring interface is in contact with the palm of the driver.

In some embodiments, the second monitoring interface is in contact with the palm of the driver.

In some embodiments, the first monitoring interface and the second monitoring interphase are in contact with the palms of the driver.

In some embodiments the first monitoring interface and/or the second monitoring interface are is continuous or intermittent contact with the palms of the driver.

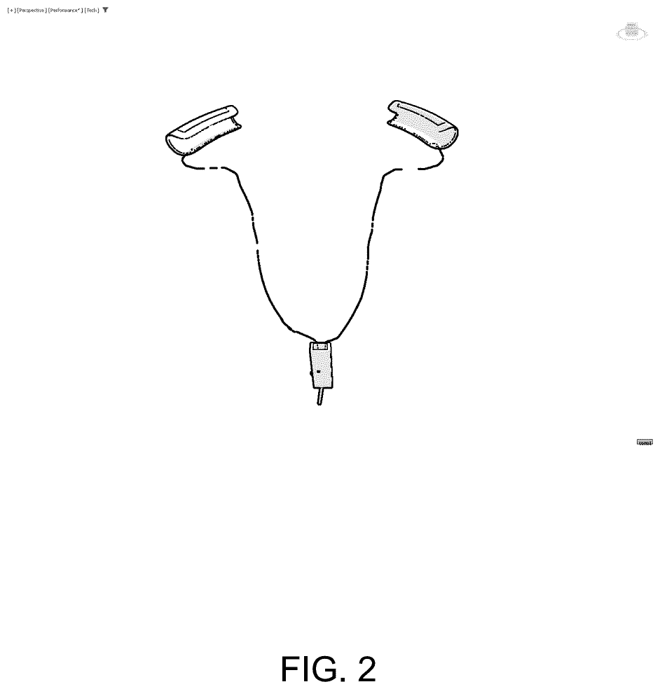

According to an aspect of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel; wherein the biased first housing and the biased second housing expose the first monitoring interface and the second monitoring interface against a palm of a subject. Referring here to FIG. 2, which shows a schematic representation of the medical device comprising first monitoring interface with sensor, second monitoring interface with sensor and subsidiary/processor module configured to be coupled to a steering wheel.

In some embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing expose the first monitoring interface and the second monitoring interface against a palm of a subject, and wherein operatively associated with the first monitoring interface and the second monitoring interface and contained in the first housing and the second housing are two or more physiological monitor units.

In some further embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing expose the first monitoring interface and the second monitoring interface against a palm of a subject wherein operatively associated with the first monitoring interface and the second monitoring interface and contained in the first housing and the second housing are two or more physiological monitor units; and wherein the first monitoring interface and the second monitoring interface are operatively linked by a processor module.

In some further embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel of a vehicle; wherein the biased first housing and the biased second housing urge the first monitoring interface and the second monitoring interface against a palm of a driver, wherein operatively associated with the first monitoring interface and the second monitoring interface and contained in the first housing and the second housing are two or more physiological monitor units; where the first monitoring interface and the second monitoring interface are operatively liked by a processor module; and wherein the processor module is coupled to the vehicle.

In some embodiments of the present invention there is provided a medical device comprising: a first monitoring interface contained in a first housing and a second monitoring interface contained in a second housing; the first monitoring interface and the second monitoring interface being operatively linked; the first housing and the second housing being configured to be biased against a steering wheel; wherein the biased first housing and the biased second housing urge the first monitoring interface and the second monitoring interface against a palm of a subject.

In some embodiments of the present invention there is provided a medical device where operatively associated with the first monitoring interface and the second monitoring interface, and contained in the first housing and the second housing and the subsidiary module are at least two or more physiological monitor units.

In some embodiments of the present invention, where the two or more operatively associated physiological monitor units include sensors, biomarkers, or the like, when the first monitoring interface and the second monitoring interface have effective skin contact, the two or more physiological monitor devices sense or monitor physiological parameters.

In some embodiments of the present invention, the two or more operatively associated physiological monitor units sensors, biomarkers, or the like, when the first monitoring interface and the second monitoring interface have effective skin contact with the palm of a subject, the two or more physiological monitor devices sense, monitor or determine a disease or disorder.

In some embodiments of the present invention, the two or more operatively associated physiological monitor devices units, sensors, biomarkers, or the like, when the first monitoring interface and the second monitoring interface have effective skin contact with the palm of a subject, the two or more physiological monitor devices sense, monitor or determine a disease or disorder.

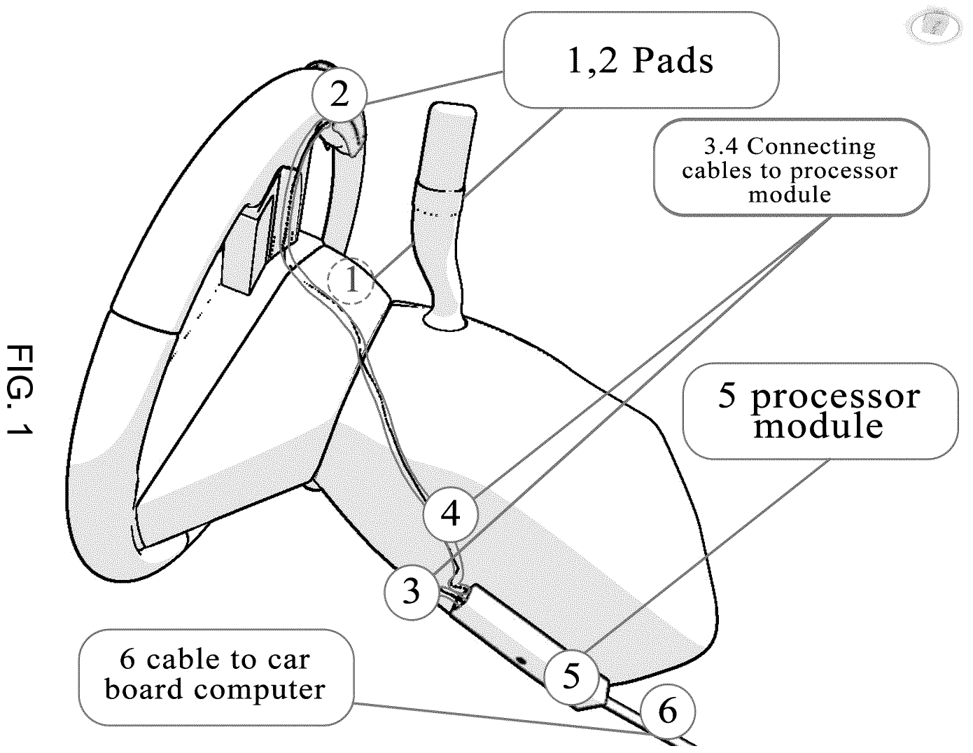

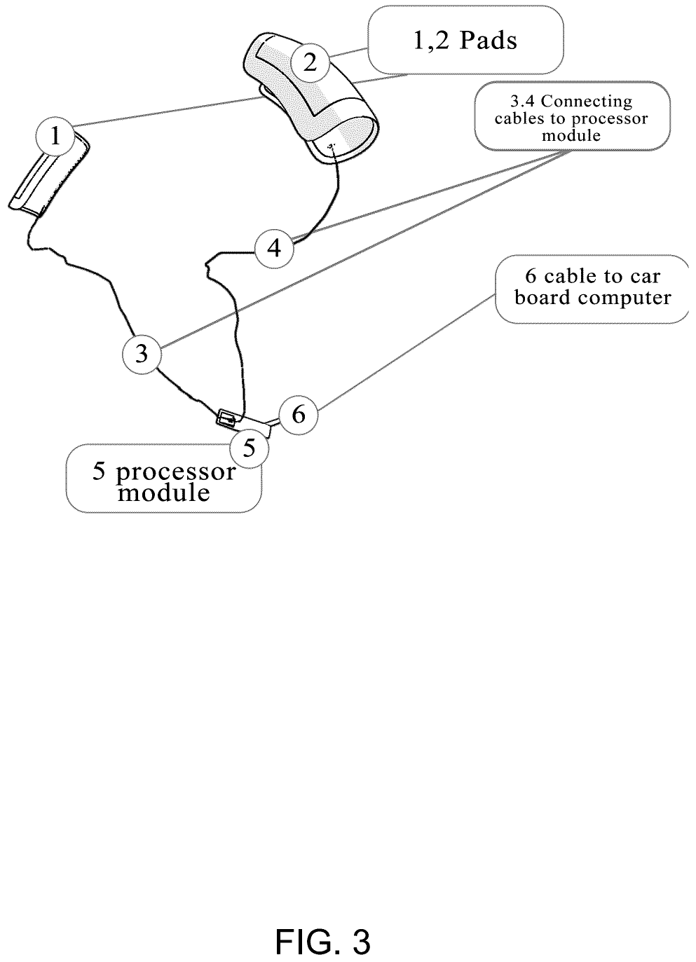

Referring to FIGS. 1 and 3, the present invention may include a medical device comprising first monitoring interface having first housing and a separate but operatively associated with the first monitoring interface a second monitoring interface comprising a second monitoring interface and a subsidiary/processor module.

In some embodiments of the present invention, the first monitoring interface and the second monitoring interface may be directly connected or communicate between each other and produce an output reading or physiological parameter determination via Bluetooth, WiFi or the like.

In some embodiments of the present invention, the first monitoring interface, the second monitoring interface and the subsidiary/processor module may be directly connected or communicate between each other and produce an output reading or physiological parameter determination via Bluetooth, WiFi or the like.

In some embodiments of the present invention, the first monitoring interface, the second monitoring interface and/or the subsidiary/processor module can communicate and thus be operatively associated by direct connection. Referring here to FIG. 1 the present invention may include a medical device comprising first monitoring interface with sensor (1), second monitoring interface with sensor (2) and subsidiary/processor module (5). Connection (3 and 4) can be used as a conduit between first monitoring interface (3) second monitoring interface (4) and the subsidiary/processor module (5).

In some embodiments of the present invention, the first monitoring interface, the second monitoring interface, the subsidiary/processor module and the control system of the vehicle, may be directly connected or communicate between each other and produce an output reading or physiological parameter determination via Bluetooth, WiFi or the like.

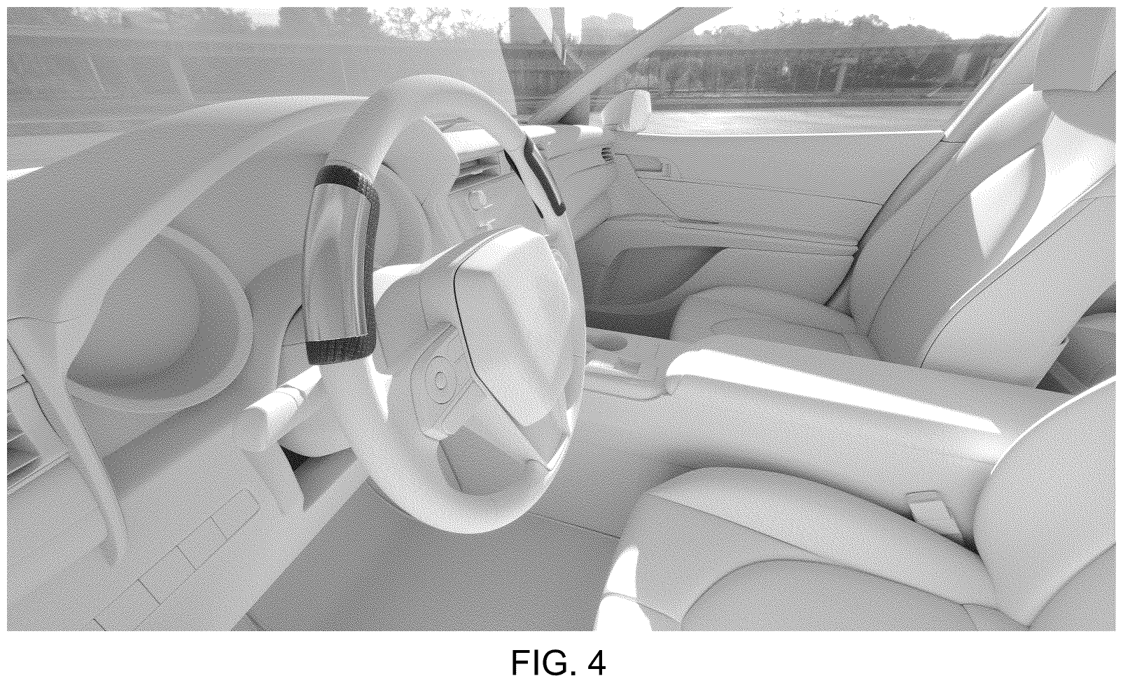

In some embodiments of the present invention, the first monitoring interface, the second monitoring interface, the subsidiary/processor module and the control system of the vehicle can communicate and thus be operatively associated by direct connection. Referring here to FIG. 1 and FIG. 4 the present invention may include a medical device comprising first monitoring interface with sensor (1), second monitoring interface with sensor (2) and subsidiary/processor module (5). Connection (3 and 4) can be used as a conduit between first monitoring interface (3) second monitoring interface (4), the subsidiary/processor module (5) and connection (6) can be used as a conduit between the processor module and the vehicle's (e.g. car or lorry) control systems (e.g. computer).

The two or more operatively associated physiological monitor units may include sensors, biomarkers, or the like, that when the first monitoring interface and the second monitoring interface have effective skin contact with the palm of a subject, the two or more physiological monitor units can sense or monitor physiological parameters.

The two or more operatively associated physiological monitor units may include sensors, biomarkers, or the like, that when the first monitoring interface and the second monitoring interface have effective skin contact with the palm of a subject, the two or more physiological monitor units can sense or monitor physiological parameters continuously.

The two or more operatively associated physiological monitor units may include sensors, biomarkers, or the like, that when the first monitoring interface and the second monitoring interface have effective skin contact with the palm of a subject, the two or more physiological monitor units can sense or monitor physiological parameters in real-time.

The two or more operatively associated physiological monitor units may include sensors, biomarkers, or the like, that when the first monitoring interface and the second monitoring interface have effective skin contact with the palm of a subject, the two or more physiological monitor units can sense or monitor predetermined physiological parameters.

The two or more operatively associated physiological monitor units may include sensors, biomarkers, or the like, that when the first monitoring interface and the second monitoring interface have effective skin contact with the palm of a subject, the two or more physiological monitor units can sense or monitor predetermined physiological parameters in real-time.

The two or more operatively associated physiological monitor units may include sensors, biomarkers, or the like, that when the first monitoring interface and the second monitoring interface have effective skin contact with the palm of a subject, the two or more physiological monitor units can sense or monitor predetermined physiological parameters intermittently in real-time.

The two or more operatively associated physiological monitor units may include sensors, biomarkers, or the like, that when the first monitoring interface and the second monitoring interface have effective skin contact with the palm of a subject, the two or more physiological monitor units can sense or monitor predetermined physiological parameters continuously in real-time.

The two or more operatively associated physiological monitor units may include sensors, biomarkers, or the like, that when the first monitoring interface and the second monitoring interfaces have effective skin contact with the palm of a subject, the two or more physiological monitor units can sense, monitor or determine a disease or disorder.

The two or more physiological monitor units may include sensors, biomarkers, or the like, that when the first monitoring interface and the second monitoring interface have effective skin contact with the palm of a subject, the two or more physiological monitor units can sense, monitor, determine or forecast disease or disorder.

The two or more physiological monitor units may include sensors, biomarkers, or the like, that when the first monitoring interface and the second monitoring interface have effective skin contact, the two or more physiological monitor units can sense, monitor or determine or forecast disease or disorder continuously and/or in real-time.

The two or more physiological monitor units may include sensors, biomarkers, or the like, that when the first monitoring interface and the second monitoring interface have effective skin contact with the palm of a subject, the two or more physiological monitor devices sense, monitor or determine the attention span, fitness and general wellness of the subject.

In some embodiments of the present invention, there is provided a medical device, comprising a monitoring interface having a housing, the housing being configured to be biased against a steering wheel of a vehicle, wherein the biased housing exposes the monitoring interface against a palm of a subject.

The present inventors have surprisingly observed that when using the physiological device of the present invention it was possible to increase the alertness of a subject such as driver or a haulier. In addition, the present inventors have surprisingly observed that when using the physiological device of the present invention it was possible to increase the duration of increased alertness of a subject such as driver or a haulier. It would be appreciated by the those in the art that an increased alertness and concomitantly the duration of the alertness offers greater safety not only to the public but also the driver. Consequently, these advantages offer a tangible and direct financial benefit.

In some embodiment, the biasing component comprised in the first housing and/or the second housing may be composed out of a resilient material such as elastic, thermoplastic, rubber or spring. The dimensions such as length, width and general size of the first housing and the second housing would be adapted depending on factors such as for example the nature of the biasing surface, the shape or form of the object. Preferably, the biasing object is a steering wheel.

Generally speaking, configuration of the biasing components are intended to hold the first monitoring interface and the second monitoring interface to allow effective skin contact with the palm of the subject.

In some embodiments of the present invention, the first monitoring interface and the second monitoring interface may be parallel to each other. Here we refer to FIG. 4, showing a steering wheel mounted medical device comprising first monitoring interface with sensor and second monitoring interface with sensor. The subsidiary/processor module is not visible. In some embodiments, the connectivity between the first monitoring interface and the subsidiary/processor module and the second monitoring interface and the subsidiary/processor module and/or the vehicle's control systems (e.g. on-board computer) can be achieved with Bluetooth, WiFi or the like. In some embodiments, the vehicle's control systems (e.g. on-board computer) can be adapted or configured to perform the operations or functions of the subsidiary/processor module.

As used in this application, the term “about” or “approximately” refers to a range of values within plus or minus 10% of the specified number. By way of non-limiting example, the term “about ten (10)” would encompass nine (9) to eleven (11) or 9-11. The term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel or substantially parallel. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

Generally speaking, subjects may be exposed to different monitoring equipment. The monitoring equipment may be for example general wellness/fitness type of monitoring equipment such as different smart watches or medical monitoring equipment such as ICU equipment. The boundaries between the general wellness/fitness monitoring equipment and the medical monitoring equipment is becoming more and more overlapping as a result of the ever, increasing understanding and appreciation of the physiologically relevance different bodily functions and biomarkers play in both wellness/fitness and the general health status of a subject during driving.

The device of the present invention may be used to measure important bodily functions and parameters which are relevant for the attention span, fitness and general wellness of a subject during driving, such as for example heart rate, pulse rate, blood pressure, blood volume, oxygen levels, sugar levels, blood ketone levels, purine levels, liver enzyme activity and haemoglobin levels.

In some embodiments of the present invention, the device is configured to measure or monitor at least one of the group comprising heart rate, pulse rate, blood pressure, blood volume, oxygen levels, sugar levels, blood ketone levels, purine levels, liver enzyme activity and haemoglobin levels on a subject during driving.

In some embodiments of the present invention, the device is configured to measure or monitor continuously, at least one of the group comprising heart rate, pulse rate, blood pressure, blood volume, oxygen levels, sugar levels, blood ketone levels, purine levels, liver enzyme activity and haemoglobin levels on a subject during driving.

In some embodiments of the present invention, the device is configured to measure or monitor intermittently, at least one of the group comprising heart rate, pulse rate, blood pressure, blood volume, oxygen levels, sugar levels, blood ketone levels, purine levels, liver enzyme activity and haemoglobin levels on a subject during driving.

In some embodiments of the present invention, the device is configured to measure or monitor continuously or intermittently in real-time, at least one of the group comprising heart rate, pulse rate, blood pressure, blood volume, oxygen levels, sugar levels, blood ketone levels, purine levels, liver enzyme activity and haemoglobin levels on a subject during driving.

A subject can also be exposed to different types of monitoring equipment to measure and monitor for example biomarkers and/or parameters such as one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

Data collected by the medical device of the present invention can be processed and presented as mean standard deviation (mSD).

In some embodiments of the present invention, the medical device is configured to measure or monitor biomarkers and/or parameters such as one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

In some embodiments of the present invention, the medical device is configured to measure or monitor continuously biomarkers and parameters such as one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

In some embodiments of the present invention, the medical device is configured to measure or monitor continuously or intermittently in real-time biomarkers and parameters such as one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

In some instances, sensing devices may be used for pulse oximetry, which may be an effective and quick way to monitor heart and lung function of a subject. These pulse oximetry devices may be capable of evaluating the colour of blood as the amount of oxygen carried by the haemoglobin may affect the colour of blood. In some examples, a pulse oximetry device may be placed on a wearer to measure the oxygenation of the person's blood.

In some embodiments, the device of the present invention is configured to monitor, such as continuously or intermittently monitor in real-time, partial pressure of oxygen PaO₂ of a subject such as a driver or a haulier during driving.

In some embodiments, the device of the present invention continuously or intermittently monitors partial pressure of oxygen PaO₂ of a subject such as a driver or a haulier during driving.

In some embodiments, the device of the present invention monitors, such as continuously or intermittently in real-time monitors, oxygen saturation SpO₂ of a subject such as a driver or a haulier during driving.

In some embodiments, the device of the present invention continuously or intermittently monitors oxygen saturation SpO₂ of a subject such as a driver or a haulier during driving.

Monitoring, such as continuous monitoring or continuous monitoring in real-time of cardiac rhythm, is also contemplated in the present invention which can enable transformative diagnostic and management tools for a driver or a haulier during driving.

In some embodiments, sensors are incorporated in the device allowing continuous monitoring of cardiac rhythm.

In some embodiments, optical sensors are incorporated in the device allowing continuous monitoring of blood volume variations referred to as photoplethysmography (PPG) from which the heart rate and other physiological parameters can be extracted to inform about the attention span, alertness, fitness and general wellness of the subject. In some embodiments, optical sensors are incorporated in the device allowing continuous monitoring of blood volume variations referred to as photoplethysmography (PPG) from which the heart rate and other physiological parameters can be extracted to inform about the attention span, alertness, fitness and general wellness of a driver such as a haulier.

In some embodiments, the device of the present invention monitors cardiac rhythm. In some embodiments, the device of the present invention monitors cardiac rhythm. In some embodiments, the device of the present invention continuously monitors cardiac rhythm.

In some embodiments, the medical device of the present invention monitors PPG. In some embodiments, the device of the present invention monitors PPG. In some embodiments, the device of the present invention continuously monitors PPG. In some embodiments, the device of the present invention continuously in real-time monitors PPG.

In some embodiments, sensors are incorporated in the medical device of the present invention allowing continuous monitoring of a subject's pulse. In some embodiments, sensors are incorporated in the medical device of the present invention allowing continuous monitoring of a subject's pulse rate.

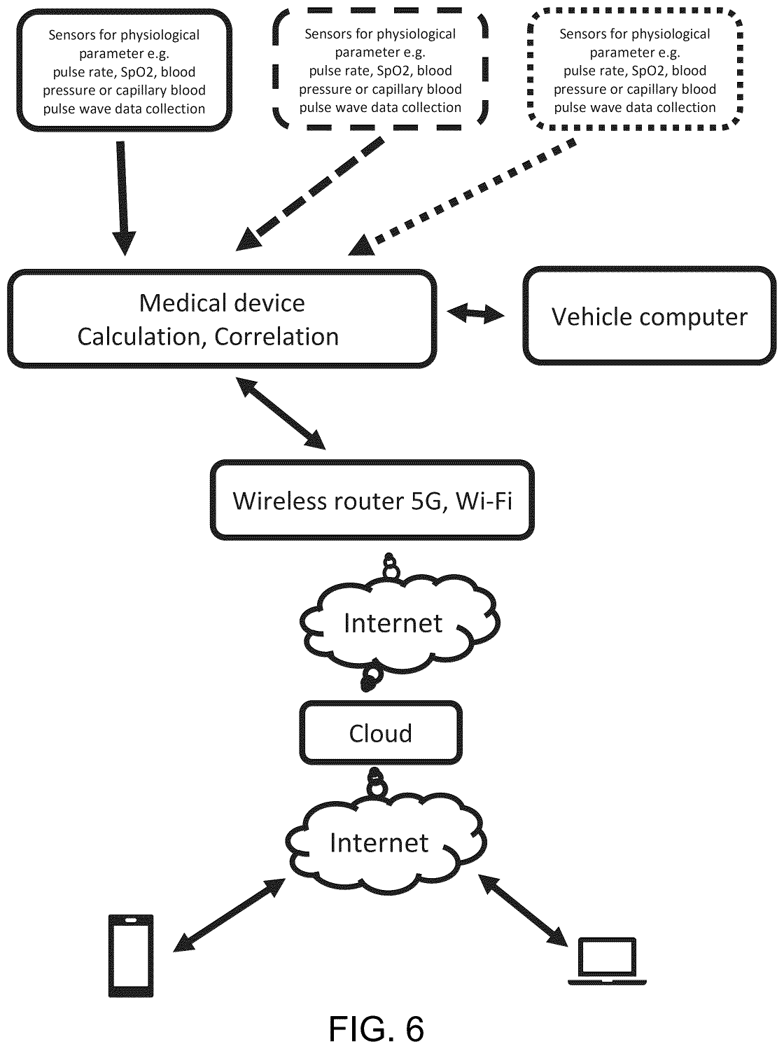

Referring here to FIG. 6 and without wishing to be bound by theory, a delay in pulse transit time (PTT) between the capillary blood pulse wave and arterial blood pulse wave has a direct link to changes in blood pressure. In other words, the ability to detect accurately and interpret meaningfully the delay observed at for example two separate points of reference on a subject between the capillary blood pulse wave and arterial blood pulse would be informative of the blood pressure of the subject such as a driver or a haulier during driving.

In some embodiments, sensors are incorporated in the medical device of the present invention allowing continuous monitoring of a subject's PTT of a subject such as a driver or a haulier during driving. In some embodiments, the first monitoring interface and the second monitoring interface sense, monitor or determine the capillary pulse wave and the arterial pulse wave of a subject such as a driver or a haulier during driving. In some embodiments of the present invention, the capillary pulse wave and the arterial pulse wave are determined simultaneously, sequentially or concomitantly on a subject such as a driver or a haulier during driving.

In some embodiments of the present invention, the capillary pulse wave and the arterial pulse wave are determined simultaneously on a subject such as a driver or a haulier during driving. In some embodiments of the present invention the simultaneously, sequentially or concomitantly determined capillary pulse wave and the arterial pulse wave can be correlated mathematically. In some embodiments of the present invention the simultaneously determined capillary pulse wave and the arterial pulse wave are correlated mathematically. In some embodiments of the present invention, the capillary pulse wave and the arterial pulse wave are correlated using computational software analysis. The computational software analysis can be performed by the on-board computer of the vehicle. In some embodiments of the present invention, the capillary pulse wave and the arterial pulse wave are correlated using artificial intelligence (AI). In some embodiments of the present invention, the capillary pulse wave and the arterial pulse wave are correlated using self-learning AI.

In some embodiments, sensors are incorporated in the medical device of the present invention allowing continuous monitoring of blood pressure. In some embodiments, sensors are incorporated in the medical device of the present invention allowing continuous monitoring of blood pressure in real-time.

### Physiological Monitor Devices

The skilled person in the art would appreciated that different physiological monitor units can be incorporated in the medical device of the present invention. It is contemplated that at least two physiological monitor units can be incorporated in the medical device of the present invention.

The physiological monitor units can be sensors, biomarkers, or the like and can be incorporated in the medical device to monitor or measure for example physiological parameters continuously or continuously in real-time on a subject. In some embodiments, the device comprises an electrode sensor.

In some embodiments, the device comprises an optical sensor. In some embodiments, the device comprises a light-emitting diode (LED). In some embodiments, the LED comprises a near-infrared LED.

In some embodiments, the device comprises a photodiode. In some embodiments, the device comprises a laser. In an embodiment, the device comprises two or more from the group consisting or an electrode sensor, an optical sensor, light-emitting diode (LED) sensor, a near-infrared LED sensor, a photodiode sensor and a laser.

In some embodiments, the main housing and the subsidiary module may each include a strain gauge-based semiconductor thin film sensor, a capacitive thin film sensor for detecting capacity change according to a pressure, a piezoresistive sensor using a piezo resistance effect, or other various pressure sensors.

Without wishing to be bound by theory, the choice of physiological monitor device may be influenced by different factors such as for instance the type of physiological biomarker or parameter which is to be monitored or measures and in particular which parameters are of relevance to the subject such as the driver or haulier during driving.

Other contributory factors to the choice of physiological monitor device include proximity of the blood vessels to the skin surface around the first monitoring interface and the subsidiary module. The accurate measurement of blood pressure is essential for example the diagnosis and management of hypertension.

Human error is also a factor as well as the place where measurements are taken for example in an office setting or out and about setting with multiple measurement being taken. One of the advantages of the present medical device is the skin of the palm is glabrous (i.e. hairless) and thus offers increased blood supply and greater sensitivity. The greater blood supply provides greater reliability of the physiological measurements and determinations obtained using the medical device of the present invention.

A particular factor which may influence the choice of the physiological monitor device, especially when assessing or determining for example blood pressure is the proximity of the device with respect to the heart of the subject.

An important and impactful factor which may influence the quality of the physiological monitor device, especially when assessing or determining for example blood pressure is the relative and alignment of the monitoring interface with the heart. The present invention offers a particular advantage since the first monitoring interface and the second monitoring interface are relatively level with the position of the heart of the subject such as the driver or the haulier during driving. The arrangement offers better quality and reliability of the physiological measurements obtained by the medical device of the present invention.

The proximity to the heart may facilitate more accurate measurements when detecting or monitoring physiological biomarkers or parameters such as for instance heart rate, pulse, blood oxygen level, blood pressure, capillary pulse wave or arterial pulse wave.

A further factor which may influence the choice of the physiological monitor include the distance between the first monitoring interface and the second monitoring surface. In order to increase accuracy of the measurements the first monitoring interface and the second monitoring surface can be configured to slide or move along the steering wheel of the vehicle. The distance or juxtaposition of the first monitoring interface and the second monitoring surface may thus freely be modified or adjusted by the subject to achieve the desired nominal contact with the skin of the palm of the subject.

Current blood pressure (BP) measurement devices employ an inflatable cuff and thus cannot be used anytime or anywhere to manage for example hypertension. Pulse transit time (PTT) varies inversely with BP in a person due to the physical properties of arteries and can be obtained without a cuff. As a result, PTT is being widely pursued for cuff-less BP measurement. The present invention provides a medical device which is capable of cuff-less BP measurement which is easy to use and accurate.

Additionally, the physiological monitor medical device of the present invention may be useful for controlled environments. The device may be particularly useful for monitoring daily routine activities, simulator training or during driving.

In particular, the physiological monitor medical device would allow richer data source to be accessed, leading to more accurate monitoring of physiological biomarkers or parameters and confidence in the data especially blood pressure monitoring during driving. Allowing quicker reaction time and intervention.

The present inventors have surprisingly observed that the medical device of the present invention provides an easy to use, consistently accurate, continuous and reliable blood pressure determination when there is achieved both a nominal contact force between the first monitoring interface and the second monitoring interface with skin of the palm of the subject such as a driver or haulier.

### Data Processing

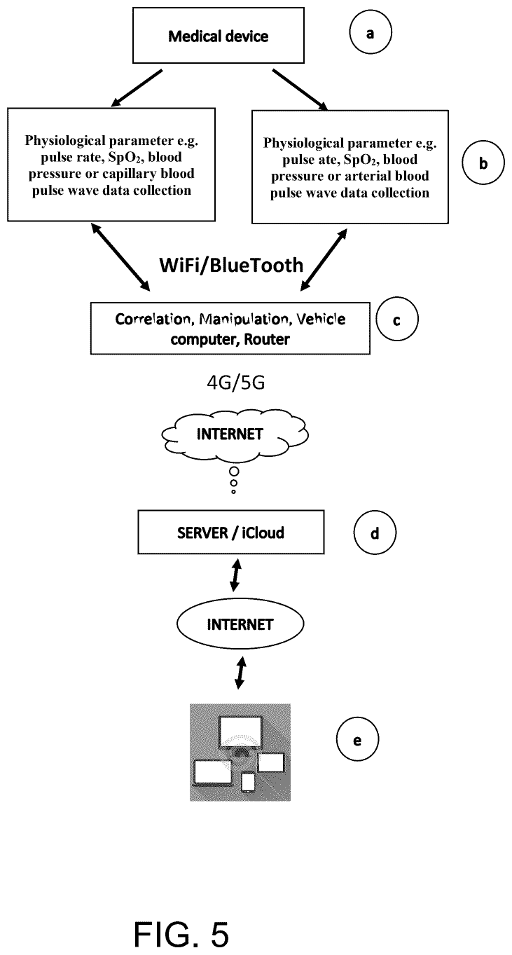

In some embodiments, the data collected from the device has to be processed on the device or remotely. Reference here is made to FIG. 5, providing a chematic representation of medical device system (a) readings or data collection (b), correlation between physiological parameter measurements such as pulse rate, SpO₂, blood pressure, capillary blood pulse wave and capillary blood pulse wave data, mathematical manipulation and software analysis of the data e.g. by vehicle's onboard computer system (c), transmittal and storage of the data on a Server/iCloud/vehicle's computer (d), and downstream uses of the collated physiological data (e).

After normalisation and pre-processing of the data, MetIDQ™ software (Biocrates) can be used for peak integration and calculation of biomarkers. If the measurements were outside the measurable range, values were imputed as follows: concentrations below the detection limit (LOD) was set to half of the lowest measured concentrations. Concentrations below the limit of quantification (LOQ) can be set to half of the LOQ. In addition, concentration higher than the highest calibration standard concentration can be set to the highest standard concentrations. In some further embodiments, the data collected from the device has to be processed using machine learning and simulated training data.

The present invention recognises that personal information data, including the physiological biomarker data acquired using the physiological monitor device of the present invention, can be used for the benefit of the wearer and/or others.

Further, other uses for personal information data, including biometric data that benefit the user are also contemplated by the present disclosure.

The present disclosure further contemplates that the entities that may be responsible for the collection, analysis, disclosure, transfer, storage, or other use of any personal information data will comply with well-established privacy policies and/or privacy practices. In particular, such entities should implement and consistently use privacy policies and practices that are generally recognised as meeting or exceeding industry or governmental requirements for maintaining personal information data private and secure, including the use of data encryption and security methods that at least meets or may even exceed industry or government standards.

An embodiment of the present disclosure may also be implemented in the form of a recording medium including instructions executable by a computer, such as a program module executed by the computer. Computer-readable media may be any available media that may be accessed by a computer and include both volatile and nonvolatile media and removable and non-removable media. In addition, the computer-readable media may include all computer storage media. The computer storage media includes both volatile and nonvolatile media and removable and non-removable media implemented by any method or technology of storing information, such as a computer readable instruction, a data structure, a program module, and other data.

### Statistical Analysis

Analysis can be carried out on the device itself or remotely using systems and software programs known in the art such as for example IBM SPSS version 25. Variables in measurement and data with skewed distributions can be log-transformed to ensure normality. Comparisons can be performed with t-test, Wilcoxon-Mann-Whitney, and one-way ANOVA as appropriate. Significance was defined as p<0.05. Non-parametric tests were used for comparing ordinal or non-normal variables. Data can be presented as mean standard deviation (mSD).

### Definitions

As used herein, the term “biomarker” refers to a physiological characteristic or physiological parameter that can be objectively measured by for example by known sensors and evaluated for example by using known statistical methods, as an indicator of normal and/or abnormal or disease processes, pharmacological responses or physiological status of a subject such as a drivee or a haulier. A “biomarker” can be used to measure the onset or the progress of a disease, the effects of treatment or regimen, or provide information on user activity, fitness, alertness, focus, attention, health and metabolic status. One of the advantages of the medical device described herein is that biomarker measurements can be collected without disruption of the skin or direct contact with the blood supply of the wearer.

In some embodiment, the biomarker is a predetermined physiological parameter.

In some embodiments, the parameter can be chemical, physical or biological.

The parameter can be altered or modified where the alteration can be measured or evaluated continuously or continuously in real-time by the device in situ i.e. while in place on a subject.

The parameter can be altered or modified where the alteration can be measured or evaluated continuously or continuously in real-time by the device remotely.

In some embodiments, the biomarker or parameter can be selected from one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; pulse transit time (PtT), pulse; blood pressure; capillary pulse wave; arterial pulse wave and C-reactive protein (CRP).

As used herein, the term “alteration” may be used interchangeably with the terms, “alter” or “modify” such as increase or decrease in the level of a metabolite such as a chemical or a biomarker detected and/or analysed and/or monitored, by the device of the present invention.

In some embodiments, the alteration or delay is at least 0.001%, 0.005%, 0.01%, 0.05%, 0.08%, 0.1%, 0.15%, 0.2%, 0.4%, 1%, 2%, 2.5%, 5%, 10%, 15%, 20%, 25%, 30%, 35%, 40%, 45%, 50%, 55%, 60%, 65%, 70%, 75%, 80%, 85%, 90%, 95% or 100% or greater compared to control or base level.

In some embodiments the alteration or delay may be at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or base level.

In further embodiments, the present disclosure may also be implemented in the form of a recording medium including instructions executable by a computer, such as a program module executed by the computer. Computer-readable media may be any available media that may be accessed by a computer and include both volatile and nonvolatile media and removable and non-removable media. In addition, the computer-readable media may include all computer storage media. The computer storage media includes both volatile and nonvolatile media and removable and non-removable media implemented by any method or technology of storing information, such as a computer readable instruction, a data structure, a program module, and other data. The storage may be in the iCloud.

Although the device and system according to the present disclosure are described with reference to specific embodiments, some or all of their components or operations may be implemented by using a computer system having a general-purpose hardware architecture such as those fitted in a vehicle such as a car.

As used herein, “treating” or “treatment” of any disease, disorder or alertness level, in certain embodiments, to ameliorating a disease or disorder that exists in a subject such as a driver or a haulier. “Treating” or “treatment” includes ameliorating at least one physiological or physical parameter such as alertness, which may be indiscernible by the subject. In yet another embodiment, “treating” or “treatment” includes modulating the disease or disorder, either physically (e.g., stabilization of a discernible symptom such as tiredness, lack of focus, dizziness, brain fog etc.) or physiologically (e.g., stabilisation of a physical parameter such as increased alertness, higher attention, greater focus etc) or both.

As used herein the terms “subject”, “driver” “haulier” are used interchangeably. As used herein, the term “subject” means a vertebrate, such as human. Preferably the subject can or is learning to drive a vehicle.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down”, “upper”, “lower”, “above”, “below”, “beneath”, “front”, “back”, “over”, “under”, “left”, “right”, “dorsal”, “volar”, “palm side”, “back side” etc. are used with reference to the orientation of some of the components of the medical device of the present invention. Since constituents or components in various embodiments described here can be positioned in a number of different orientations, locations of the body, directional terminology is used for purposes of illustration only and is in no way limiting. The directional terminology is intended to be construed broadly, and therefore should not be interpreted to preclude components being oriented in different ways.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. It will also be appreciated that the device(s), method(s), use(s), detector(s), sensor(s), physiological biomarker(s) may be subject to numerous rearrangements, modifications and substitutions without departing from the scope of the present disclosure as set forth and defined by the following claims.

What is claimed is:

1. A medical device, comprising:
a first monitoring interface having a first housing;
a second monitoring interface having a second housing;
the first monitoring interface and the second monitoring interface being operatively linked;
the first housing and the second housing being configured to be biased against a steering wheel of a vehicle; and
wherein the biased first housing and the biased second housing expose the first monitoring interface and the second monitoring interface against a palm of a subject.

2. A medical device according to claim 1, wherein the first monitoring interface and the second monitoring interface are operatively linked by a subsidiary module.

3. A medical device according to claim 2, wherein the subsidiary module is coupled to the vehicle.

4. A medical device according to claim 1, wherein operatively associated with the first monitoring interface and the second monitoring interface, and contained in the first housing and the second housing and the subsidiary module are at least two or more physiological monitor units.

5. A medical device according to claim 4, wherein the two or more operatively associated physiological monitor units include sensors, biomarkers, or the like, that when the first monitoring interface and the second monitoring interface have effective skin contact with the palm of a subject, the two or more physiological monitor devices sense or monitor physiological parameters.

6. A medical device according to claim 4, wherein the two or more operatively associated physiological monitor units include sensors, biomarkers, or the like, that when the first monitoring interface and the second monitoring interfaces have effective skin contact with the palm of a subject, the two or more physiological monitor devices sense, monitor or determine a disease or disorder.

7. A medical device according to claim 6, wherein the two or more operatively associated physiological monitor devices units include sensors, biomarkers, or the like, that when the first monitoring interface and the second monitoring interfaces have effective skin contact with the palm of the subject, the two or more physiological monitor devices sense, monitor or determine a disease or disorder.

8. A medical device according to claim 4, wherein the two or more operatively associated physiological monitor units include sensors, biomarkers, or the like, that when the first monitoring interface and the second monitoring interfaces have effective skin contact with the palm of a subject, the two or more physiological monitor devices sense, monitor or determine a disease or disorder continuously and/or in real-time.

9. A medical device according to claim 4, wherein the two or more operatively associated physiological monitor units include sensors, biomarkers, or the like, that when the first monitoring interface and the second monitoring interface have effective skin contact with the palm of a subject, the two or more physiological monitor devices sense, monitor or determine the attention span, alertness, fitness and general wellness of the subject.

10. A medical device according to claim 1, wherein the biomarker is one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time; hypoxanthine; pulse transit time (PTT); pulse; blood pressure; capillary pulse wave; arterial pulse wave and C-reactive protein (CRP).

11. A medical device according to claim 1, wherein the biomarker is partial pressure of oxygen PaO₂.

12. A medical device according to claim 1, wherein the biomarker is oxygen saturation SpO₂.

13. A medical device according to claim 1, wherein the biomarker is cardiac rhythm.

14. A medical device according to claim 1, wherein the biomarker is pulse transit time (PTT).

15. A medical device according to claim 1, wherein the biomarker is pulse rate.

16. A medical device according to claim 1, wherein the biomarker is blood pressure.

17. A medical device according to claim 1, wherein the first monitoring interface and the second monitoring interface sense, monitor or determine the capillary pulse wave and the arterial pulse wave.

18. A medical device according to claim 17, wherein the capillary pulse wave and the arterial pulse wave are determined simultaneously, sequentially or concomitantly.

19. A medical device according to claim 1, wherein a subject is monitored continuously.

20. A medical device, comprising:
a monitoring interface having a housing;
the housing being configured to be biased against a steering wheel of a vehicle; and
wherein the biased housing exposes the monitoring interface against a palm of a subject.
